(19) United States
(12) Patent Application Publication
(10) Pub. No.: US 2012/0197187 A1
(43) Pub. Date: Aug. 2, 2012

(54) BREAST PUMP SYSTEM

(76) Inventor: Julie LaFave, (US)

(21) Appl. No.: 13/357,928

(22) Filed: Jan. 25, 2012

Related U.S. Application Data
(60) Provisional application No. 61/437,421, filed on Jan. 28, 2011.

(51) Int. Cl. A61M 1/06; A41C 5/00; A41C 3/08

(52) U.S. Cl. 604/74; 450/31; 450/92

(57) ABSTRACT

The present disclosure provides for a hands-free battery operated breast pump system having a battery operated breast pump, tubing, connector pieces, flask-type storage containers, funnels, a stretch bra and a vest with pockets. The vest provides covers the mother and baby sufficiently to provide privacy during a breastmilk expressing process such that the breast is not outwardly exposed, while also allowing the mother to perform other tasks unhindered while expressing breastmilk for storage.

### CROSS REFERENCE TO RELATED APPLICATIONS

This is a non-provisional and claims benefit of U.S. Provisional Application No. 61/437,421 filed on Jan. 28, 2011, which is hereby incorporated by reference in its entirety.

### FIELD

The present document provides a breast pump system, and in particular to a breast pump system having a vest or jacket operatively associated with a breast pump that provides complete coverage to a lactating woman during a breast milk expressing process such that the breasts of the woman are not exposed, while also allowing her to perform other tasks when expressing breast milk.

### BACKGROUND

A nursing mother (or one who expresses milk exclusively), may use her breast pump every two to three hours for a year or more. Discounting the night hours, it is easy to conclude a woman may average three to six 20-30 minute pumping sessions per day (with variations for personal use/need). Assuming four 20 minute sessions per day, a woman could easily spend one and a half hours per day (as a conservative estimate) connected to the breast pump. Conservatively, a woman could spend 10 hours a week and close to 500 hours in the baby's first year, connected to her breast pump. While there are currently hands-free breast pump devices or systems available, they are cumbersome to use and difficult to assemble. In addition, although conventional breast pump devices may use clips to engage the breast pump device to an existing bra worn by the woman, such a clip arrangement can also pull down on the breast during pumping operation, which can cause leakage of breast milk and/or disconnection of the breast itself from the breast pump device. Therefore there is a need for a breast pump system which allows a lactating woman to maximize her time when expressing breast milk in order to be more productive by being able to concurrently perform other duties around the house or out in the public.

### SUMMARY

In an embodiment, a breast pump system may include a garment including an interior portion and an exterior portion with the garment defining a first sleeve opening and a second sleeve opening configured to permit passage of an individual's arms. The breast pump system further includes a bra secured to the garment with the bra having a first bra portion defining a first aperture and a second bra portion defining a second aperture, wherein the first bra portion and the second bra portion are positioned along the interior portion of the garment proximate the first sleeve opening and the second sleeve opening, respectively.

In another embodiment, a method of manufacturing a breast pump system may include:
providing a garment material including an interior portion and an exterior portion,
forming a first sleeve opening and a second sleeve opening in the garment material configured to permit passage of an individual's arms;
securing a bra having a first bra portion and a second bra portion to the interior portion of the garment material; and
forming a first opening through the first bra portion and a second opening through the second bra portion.

Additional objectives, advantages and novel features will be set forth in the description which follows or will become apparent to those skilled in the art upon examination of the drawings and detailed description which follows.

### BRIEF DESCRIPTION OF THE DRAWINGS

FIG. 1 is a front view of a vest used in a breast pump system showing the interior portion of the vest;

FIG. 2 is a front view of the vest with the vest in a partially closed configuration.

FIG. 3 is a rear view of the vest;

FIG. 4 is a side view of a collection member used with the vest of the breast pump system;

FIG. 5 is a front view of one embodiment for the breast pump connected to a dual tubing arrangement of the breast pump system;

FIG. 6 is a front view of the breast pump system showing a pair of collection members operatively connected to the vest;

FIG. 7 is a front view showing another embodiment of the vest configured to engage a different embodiment of the breast pump;

FIG. 8 is a front of a jacket used with another embodiment of the breast pump system; and

FIG. 9 is a front view of another embodiment of the breast pump connected to a single tubing arrangement of the breast pump system.

Corresponding reference characters indicate corresponding elements among the view of the drawings. The headings used in the figures should not be interpreted to limit the scope of the claims.

### DETAILED DESCRIPTION

The detailed description herein describes for systems and methods for a breast pump system that ensures a degree of privacy during the milk expressing period by a lactating woman. The breast pump system allows a lactating woman to walk and/or perform other tasks during the milk expressing process for collecting expressed breast milk. In some embodiments, the breast pump system may comprise a garment, such as a vest or jacket, that engages a bra for supporting a breast pump, tubing arrangement, and one or more collection members for collecting expressed breast milk as well as provide substantial coverage of a lactating woman's upper body, especially her breasts during the milk expressing process. As such, the breast pump system described herein allows a lactating woman to recapture some of the nursing time required to express milk and be more productive with her time since the garment and bra arrangement for a breast pumping system provides a degree of privacy to the user when expressing breast milk, allows for unencumbered use in operating the breast pump system, and permits hands-free operation of the breast pump.

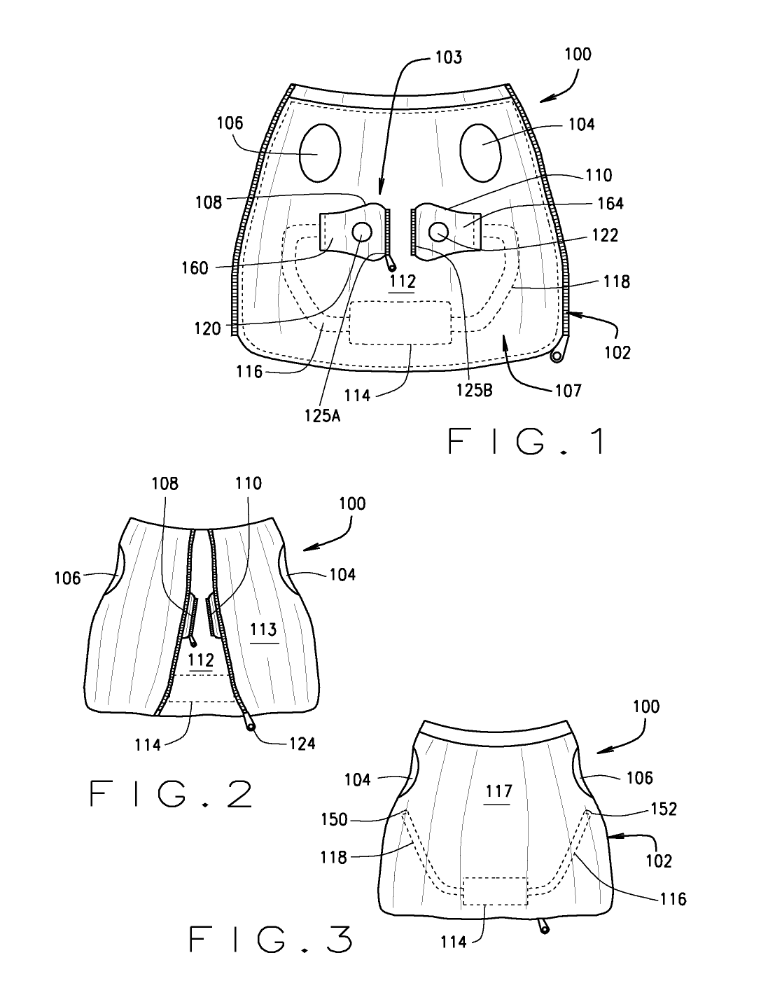

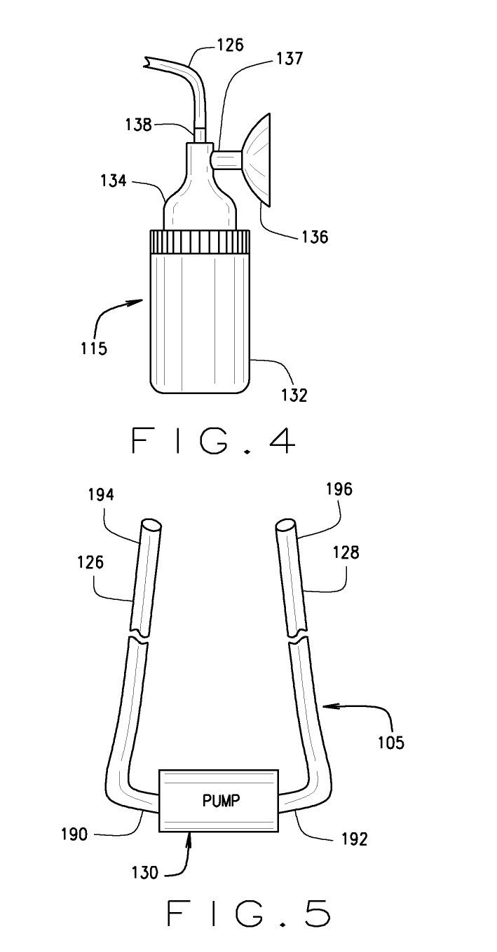

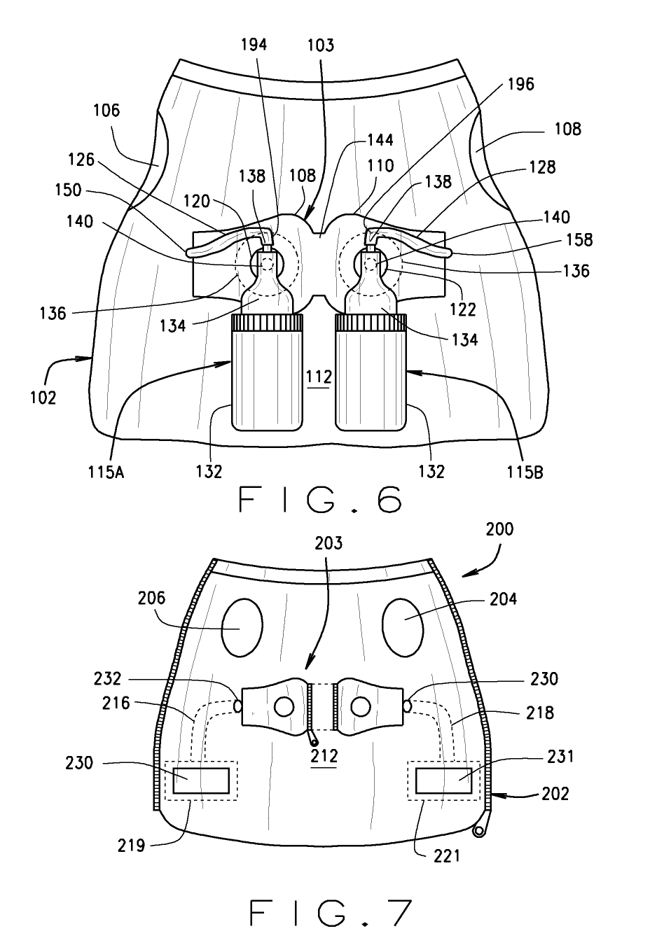

Referring to the drawings, various embodiments of the breast pump system are illustrated and generally indicated as 100 200 and 300 in FIGS. 1-9. In one embodiment shown in FIG. 1, the breast pump system 100 may include an outer garment, such as a vest 102 configured to receive a breast pump 130 (FIG. 5) connected to a dual tubing arrangement 105 having a first tube 126 and a second tube 128 that are each configured to be connected to respective collection members 115A and 115B (FIG. 4) for collecting breast milk expressed from a lactating woman. The vest 102 defines a first sleeve opening 104 and a second sleeve opening 106 configured to permit the lactating woman's arms to pass through when the vest 102 is being worn.

As shown in FIGS. 1-3, one embodiment of the vest 102 includes an interior portion 112, such as an inner liner, connected or otherwise sewn to a front exterior portion 113 and a rear exterior portion 117 that form the outside portions of the vest 102. In some embodiments, the interior portion 112 may be made from a flexible material, such as fabric or plastic material, connected or otherwise sewn to the front and rear exterior portions 113 and 117 of the vest 102 to form an inner space 107 between the interior portion 112 and exterior portions 113 and 117.

In the embodiment shown in FIG. 1, a pocket 114 may be formed along the vest 102, which communicates with a first channel 116 and a second channel 118 in which the pocket 114 and channels 116 and 118 are sewn or otherwise formed along the interior portion 112 and/or exterior portions 113 and 117 of the vest 102. The pocket 114 may be configured to receive and store the breast pump 130 therein and the first and second channels 116 and 118 may be configured to receive the dual tubing arrangement 105 with the first tube 126 inserted through the first channel 116 and the second tube 128 inserted through the second channel 118 in one method of assembly.

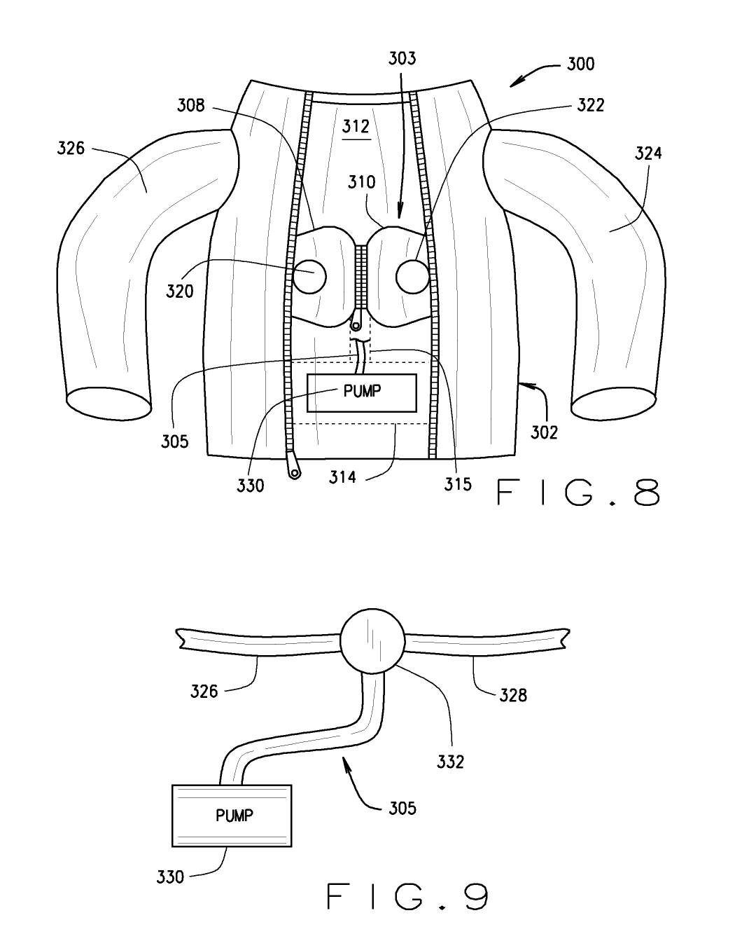

Referring to FIG. 7, one embodiment of the breast pump system, designated 200, includes a vest 202 which is similar to vest 102 in that the vest 202 defines a first sleeve opening 204 and a second sleeve opening 206 along an interior portion 212. In some embodiments, the vest 202 may include first pocket 219 and a second pocket 221 positioned on either side of vest 202. In some embodiments, the first pocket 219 may be configured to receive a first breast pump 230 and the second pocket 221 configured to receive a second breast pump 231 for expressing breast milk into first and second collection members 115A and 115B (FIG. 6). In those embodiments of the breast pump system 200 having a single breast pump 132 with a separate battery pack, the first pocket 219 may be configured to receive one of the first and second breast pumps 230 and 231, while the second pocket 221 may be configured to receive a separate battery pack for operative connection to the breast pump 230. In such an embodiment, a channel (not shown) may be formed that communicates between the first and second pockets 219 and 221 to allow passage of an electrical line from the battery pack to the breast pump 230 to provide electrical power. In addition, the first pocket 219 may communicate with a first channel 216 and the second pocket 221 may communicate with a second channel 218 for establishing a pathway for tubing arrangement 105 (FIG. 3) or tubing arrangement 305 (FIG. 9) As shown, the first channel 216 includes an opening 232 and the second channel 218 includes a second opening 230 for permitting passage of the tubing arrangements 105 or 305. In addition, the vest 202 includes a bra 203, similar to bra 103, which is configured to engage the vest 203 as well as collection members 115 for collecting expressed milk.

In another embodiment shown in FIG. 8, the breast pump system, designated 300, includes a jacket 302, which is substantially similar to the vests 101 and 202 with the exception that the jacket 302 includes sleeves 324 and 326 extending from the sleeve openings. In some embodiments, the jacket 302 is configured to engage a bra 303 having a first bra portion 308 defining a first opening 320 and a second bra portion 310 defining a second opening 322 in which the first and second bra portions 308 and 310 are coupled to the interior portion 312 of the jacket 302. In some embodiments, the jacket 302 may include a single pocket 314 configured for receiving a breast pump 330 and a single channel 315 extending from the pocket 314 for receiving tubing arrangement 305. In some embodiments, the jacket 302 may include first and second pockets in communication with respective channels as illustrated in FIG. 7. In addition, the embodiment of the tubing arrangement 305 shown in FIG. 9 includes a first tube 326 and a second tube 328 that extend from a connector 332, such as a Y-connector, for connection to respective collection members 115, while a third tube 332 extends between the connector 332 and the breast pump 330.

Referring back to FIGS. 2 and 3, one embodiment of the vest 102 may include a first channel 116 defining an opening 152, while the second channel 118 defines another opening 150, which are both configured to permit passage of the first and second tubes 126 and 128, respectively, of the tubing assembly 105 during assembly of the breast pump system 100. The first and second openings 150 and 152 may be located proximate the first and second bra portions 108 and 110, respectively. In other embodiments, the vest 102 may lack the first and second channels 116 and 118 such that the tubing arrangement 105 passes directly through the inner space 107 between the interior portion 112 and exterior portions 113 and 117 without being confined within the first and second channels 116 and 118 for passage of the first tube 126 and second tube 128 directly through first and second openings 150 and 152, respectively.

As shown in FIG. 1, the flexible bra 103 defines a first bra portion 108 and a second bra portion 110 located proximate or adjacent the first and second sleeve openings 104 and 106, respectively. In some embodiments, the first bra portion 108 includes a respective flexible body 160 defining a first opening 120 with one end of body 160 sewn or otherwise engaged to the interior portion 112 and an opposing end of body 160 having a zipper arrangement 125A. Similarly, the second bra portion 110 includes a flexible body 164 defining a second opening 122 with one end portion sewn or otherwise engaged to the interior portion 112 and an opposing end having a complementary zipper arrangement 125B configured to engage zipper arrangement 125A for securing together the first and second bra portions 108 and 110.

The first and second bra portions 108 and 110 may have any suitable configuration, such as a square-shaped configuration, a rectangular-shaped configuration, a circular-shaped configuration, an oval-shaped configuration, an irregular-shaped configuration, and combinations thereof which are sized and shaped to receive the breasts of the lactating woman when wearing the bra 103. As noted above, the first bra portion 108 and the second bra portion 110 may be separate, distinct portions sewn or otherwise engaged to the interior portion 112 proximate the first and second sleeve openings 104 and 106 of the vest 102 (FIG. 1), while in other embodiments the flexible bra 103 may be of unitary construction in which a middle portion 144 (FIG. 6) extends between the first and second bra portions 108 and 110 with a part of the middle portion 144 being engaged between the interior portion 112 and the exterior portion 117 of the vest 102 to engage the bra 103 to the vest 102.

Referring back to FIG. 2, some embodiments of the vest 102 may include the zipper arrangement 124 for securing together the front portions of the vest 102 such that the interior portion 112 of the vest 102 is not outwardly visible when the breast pump system 100 is being used. In other embodiments, the vest 102 may include various other means for securing the front of the vest 102, such as a button arrangement, a clasp arrangement, a VELCO® arrangement, a snap-on arrangement, or a tie-string arrangement. The above means for securing the front of the vest 102 may also be used to secure the first and second bra portions 108 and 110 together.

In some embodiments, the breast pump 130 may be battery operated and the battery (not shown) rechargeable. In another embodiment, the breast pump 130 may include a separate battery pack (not shown). In one embodiment, the breast pump 130 may be made into a comparable size and substantially flat in shape so that it can be housed in the pocket 114 of the vest 102. In an alternative embodiment, the breast pump 130 may be housed in one or more pockets 114 located along the interior and/or exterior portions 112, 113 and 117 of the vest 102. In one embodiment, the battery pack may be an integral part of the breast pump 130. In another embodiment, the battery pack and the breast pump 130 may be two separable components. In such an embodiment, shown in FIG. 7, the battery pack may then be housed in pocket 219 on one side of the vest 202 to balance the weight of the breast pump 130 disposed in another pocket 221 on the other side of the vest 102. In one embodiment, the breast pump 130 may be located outside the vest 102 and plugged in to an external power source, such as an electrical outlet or automobile charger.

Referring to FIG. 4, the breast pump system 100 includes at least one collection member 115 for collecting expressed breast milk. The collection member 115 includes a collection chamber 132 configured to collect expressed breast milk. The collection chamber 132 is in fluid flow communication with a manifold 134 having a port 138 configured to engage one of the first and second tubes 126 and 128 to apply a negative pressure to the collection chamber 132 produced by the breast pump 130. The collection member 115 further includes a funnel 136 connected or integral with the manifold 134. The funnel 136 is configured to receive a woman's breast during the lactating process such that sufficient suctioning action is applied to the breast for causing breast milk to be expressed into the collection chamber 132 through the funnel 136 and manifold 132. As such, engagement of the funnel 136 to the woman's breast and operation of the breast pump 130 generates sufficient negative pressure for producing a suctioning action to the lactating woman's breast to express breast milk into the collection chamber 132.

In one method of assembling the breast pump system 100 illustrated in FIGS. 5 and 6, the first and second collection members 115A and 115B are engaged to the first bra portion 108 and second bra portion 110, respectively, by inserting the neck portion 137 of each funnel 136 through respective openings 120 and 122 of first and second bra portions 108 and 110, and then engaging each funnel 136 to a respective first and second collection members 115A and 1158. Once the first and second collection members 115A and 1158 are mounted to the bra 103 in this manner, the proximal ends 190 and 192 of the dual tubing arrangement 105 may be connected to the breast pump 130 and the distal ends 194 and 196 of the dual tubing arrangement 105 may be inserted through respective openings 150 and 152 for connection to ports 138 of first and second collection members 115A and 115B, respectively, to establish fluid flow communication between the breast pump 130 and the first and second collection members 115A and 115B when applying the suctioning action to the breasts of the lactating woman. In one embodiment, the first and collection members 115A and 115B may be disposed in a pocket 114 located along the front breast area of the vest 102 that is accessible to the tubing arrangement 105.

In some embodiments of the collection member 115, the collection chamber 132, manifold 134 and funnel 136 collectively define a low profile relative to the vest 102, 202 and jacket 302. In this low profile configuration, the collection member 115 does not extend or extend substantially outwardly from the front of the vests 102, 202 and jacket 302.

In some embodiments, the breast pump 130 and the collection member 115 may be manufactured by Medela (Model Nos. 57040 and 57062) or the First Year's miPump™ breast pump system (Model No. Y4613/Y4614.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A breast pump system comprising:
a garment including an interior portion and an exterior portion, the garment defining a first sleeve opening and a second sleeve opening configured to permit passage of an individual's arms; and
a bra secured to the garment, the bra including a first bra portion defining a first aperture and a second bra portion defining a second aperture, wherein the first bra portion and the second bra portion are positioned along the interior portion of the garment proximate the first sleeve opening and the second sleeve opening, respectively.

2. The system of claim 1, further comprising:
a first collection member configured to engage the first bra portion through the first aperture and a second collection member configured to engage the second bra portion through the second aperture, wherein the first collection member and the second collection member each define a respective collection chamber;
a first tubing arrangement having a first tube configured to engage the first collection member and a second tube having a first end configured to engage the second collection member; and
a breast pump for generating a vacuum, the breast pump in communication with the tube and the second tube for applying a vacuum to the first collection member and the second collection member, respectively.

3. The system of claim 1, wherein at least one channel is formed between the interior portion and the exterior portion of the garment, wherein the at least one channel is configured to receive the first tubing arrangement and the second tubing arrangement.

4. The system of claim 2, wherein the at least one channel defines a second end in communication with at least one pocket formed along the garment.

5. The system of claim 3, wherein the breast pump is configured to be disposed within at least one pocket

6. The system of claim 2, wherein the garment is a vest.

7. The system of claim 6, wherein the garment is a jacket.

8. The system of claim 1, wherein the first bra portion is engaged to the interior portion of the vest proximate to the first sleeve opening and the second bra portion is engaged interior portion of the vest proximate to the second sleeve opening.

9. The system of claim 1, further comprising a means of engaging the first bra portion to the second bra portion.

10. The system of claim 9, wherein the means for engaging is one of a zipper arrangement, a clasp arrangement, a button arrangement and tie-string arrangement, and a snap-on arrangement.

11. The system of claim 2, wherein the first collection member and the second collection member each comprise a collection chamber in communication with a manifold and a funnel.

12. The system of claim 11, wherein each funnel defines a configuration for engaging and pulling on a nipple of a lactating individual.

13. The system of claim 1, wherein the first bra portion and the second bra portion are connected by a middle portion secured to the garment.

14. The system of claim 1, wherein the garment provides complete coverage of the lactating individual's breasts during the breast milk expressing process.

15. The system of claim 2, wherein the first tube and the second tube of the first tubing arrangement are coupled together at a connector which is coupled to a third tube in communication with the breast pump.

16. The system of claim 1, wherein the vest defines a plurality of pockets.

17. A method of manufacturing a breast pump system comprising:
providing a garment material including an interior portion and an exterior portion,
forming a first sleeve opening and a second sleeve opening in the garment material configured to permit passage of an individual's arms;
securing a bra having a first bra portion and a second bra portion to the interior portion of the garment material; and
forming a first opening through the first bra portion and a second opening through the second bra portion.

18. The method of claim 17, further comprising:
securing at least one collection member to the bra, wherein the collection member includes a collection chamber in communication with a manifold having a funnel.

19. The method of claim 17, further comprising:
securing the first bra portion to the second bra portion.

20. The method of claim 18, further comprising:
a connection means for connecting a first front portion of the garment material with a second front portion of the garment material for covering the bra, the first collection member and the second collection member.
